(19) United States
(12) Patent Application Publication
(10) Pub. No.: US 2006/0201508 A1
(43) Pub. Date: Sep. 14, 2006

(54) Self contained breathing apparatus combined duration factor for breathing systems

(76) Inventors: David E. Forsyth, Laguna Beach, CA (US); Wayne K. Miller, Fort Jones, CA (US)

Correspondence Address: DORSEY & WHITNEY LLP;INTELLECTUAL PROPERTY DEPARTMENT, 50 SOUTH SIXTH STREET, MINNEAPOLIS, MN, 55402-1498, US

(21) Appl. No.: 11/215,349

(22) Filed: Aug. 30, 2005

Related U.S. Application Data
(60) Provisional application No. 60/605,561, filed on Aug. 30, 2004.

(51) Int. Cl. A62B 7/04; A61M 16/00

(52) U.S. Cl. 128/204.26; 128/204.18; 128/204.21; 128/204.23

(57) ABSTRACT

The present claim enables a breathing system to be assessed with a minimum of attention or experience regardless of type of breathing system. This is achieved by creating an electronic control system for the breathing system which converts all available parameters which affect the ongoing use of the breathing system and converting those parameters in a common basis of time remaining of practical use on the breathing system. The electronic system then combines the common parameters in time and combines as appropriate to produce a single time remaining parameter pertaining to the breathing machine operation. This approach is applied across rebreather and open circuit, land and water based systems such that an operator may use similar skills to interpret the output indications of different types of machines.

### CROSS REFERENCE TO RELATED APPLICATION(S)

The present utility patent application claims the benefit of U.S. Provisional Application Ser. No. 60/605,561, filed Aug. 30, 2004 in the names of the present applicants, subject matter of which is incorporated herewith by reference.

### FIELD OF THE INVENTION

The present invention relates generally to respiratory methods or devices; more specifically to electric control and monitoring means for the supply of respiratory gas such as described by SCBAs (Self Contained Breathing Apparatus), SCUBAs (Self Contained Underwater Breathing Apparatus), and Semi- or Fully-Closed Circuit Rebreather systems (SCR, CCR).

### BACKGROUND OF THE INVENTION

Breathing systems exist to support life in environments that otherwise would be difficult to impossible to function in. In most, if not all cases, the breathing machine exists and is used as a means to function in a hostile environment. The implication of this fact is that any device or implementation which decreases the level of attention and/or training necessary to utilize the breathing system is of value both in terms of an increase in attention that may then be directed to the purpose at hand as well as being of value in terms of decreasing the potentially lethal effect of a inadvertent lapse of attention and/or training.

The limits of duration on a breathing system typically depend on a variety of factors based on the states and status of the breathing system itself as well as the operator and the environment being operated in. Land based SCBAs (Self Contained Breathing System) based on open circuit technology may themselves depend primarily on tank pressure for system life support duration however the environment and state of the user may become significant factors in determining overall time remaining of system use.

More complicated systems such as Mixed Gas Closed Circuit Rebreathers generally consist of an array of gas bottles, high and low pressure piping, sensors, primary and redundant control systems, automatic and manual control valves, high and low pressure regulators, and display/control devices. Together these components form a complicated system which requires a high level of skill and attention to operate and can result in many indications of multiple levels, states, and time remaining indications which must be collectively interpreted by the operator.

Current implementations of breathing systems are limited in terms of communications to the user that are either a go/no-go indication giving an indication for potentially more than one error condition but having no ability to provide information other than the fact that an error has occurred (such as an alarm buzzer or warning LED) or the communications are specific as to the state or level of a single parameter on the breathing system (such as a pressure gauge or battery voltage indicator) but do not translate the factor into time remaining. It is left to the user to convert the information into time and then combine all relevant inputs into an overall time remaining conclusion. Communications conveying information about multiple states and/or levels requires multiple indications such as multiple gauges, LEDs, text displays, etc.

In addition, different types of breathing systems typically require different sets of instruments to monitor and control the system. With this approach of combining relevant factors into a common time remaining indication, it provides the benefit not previously realized of allowing a much more common set of training and experience to be useful to those operators who may be called upon to use different breathing systems as circumstances may demand it such as Firefighters or Hazardous Material handlers.

The most desirable situation in the realm of communications between the breathing system and the operator is to reduce to the maximum extent possible, the level of operator attention required in order to assure safe communication. At the same time, the more the operator is informed in those communications about the breathing system and all parameters that affect the use of that system, the more likely that choices and actions will be made in accordance with the ideal of maximizing the likelihood of staying alive.

### SUMMARY OF THE INVENTION

The invention is summarized by considering that the operator of any type of breathing system is primarily concerned with how long can that system be safely used under current conditions. The claims are made for rebreathers, land, and water based open-circuit (SCBA, SCUBA) configurations of breathing systems that the parameters available for measurement and assessment by an electronic microcontroller system may be converted into common time based factors and that these factors may then be combined into a single time remaining factor. This method is similar regardless of type of breathing system and so greatly improves the usability and safety of each system due to the simplification of the information necessary for operation use The training required for each type of system is simplified and in addition, a greater simplicity is provided for operators which may be called upon to use different types of breathing machines. While all available information to the microprocessor is considered within the scope of the overall claims, specific claims are made with regard to the translation of power capacity, CO2 scrubber capacity, CO2 levels, gas tank capacity, PPO2 levels, system mechanical functionality, electronic hardware and firmware functionality, biometric parameters, and environmental parameters to translate these parameters into a single time factor based on effect on operational time remaining on the system.

### BRIEF DESCRIPTION OF THE DRAWINGS

FIG. 1 is a picture of an electronically controlled fully closed circuit mixed gas rebreather.

FIG. 2 is a system diagram of the entire breathing system.

FIG. 3 is a schematic block diagram of the exampled control system.

FIG. 4 is a graph of the expected time remaining factor relative to the amount of Oxygen remaining in the loop as calculated for the specific loop volume of this embodiment.

FIG. 5 is a graph of expected time remaining factor relative to the amount of capacity expected out of the 9 volt battery relative to standard and measured current demands found in normal operation.

FIG. 6 is a graph of expected time remaining factor relative to the amount of capacity expected to be consumed from the Oxygen Cylinder during normal use.

FIG. 7 is a graph of expected time remaining factor relative to the capability expected of the control system in the event of microprocessor reset events.

FIG. 8 is a graph of expected time remaining factor relative to the amount of capacity expected CO2 scrubber time remaining relative to Oxygen usage for the given scrubber capacity in the exampled embodiment.

FIG. 9 is a graph of expected time remaining factor for body and environmental temperature relative to a typical work safety mandate for hazardous environments and the work guidelines that are applied to those environments.

FIG. 10 is a flow chart of the interaction and combination of the various time remaining factors into a single combined system duration factor.

### DETAILED DESCRIPTION OF THE PREFERRED EMBODIMENT

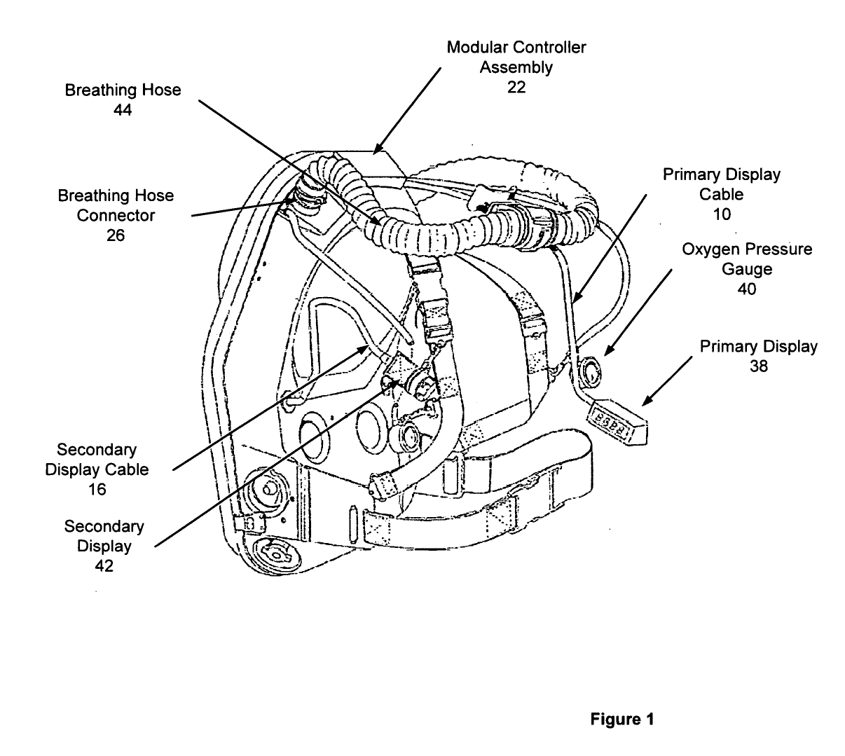

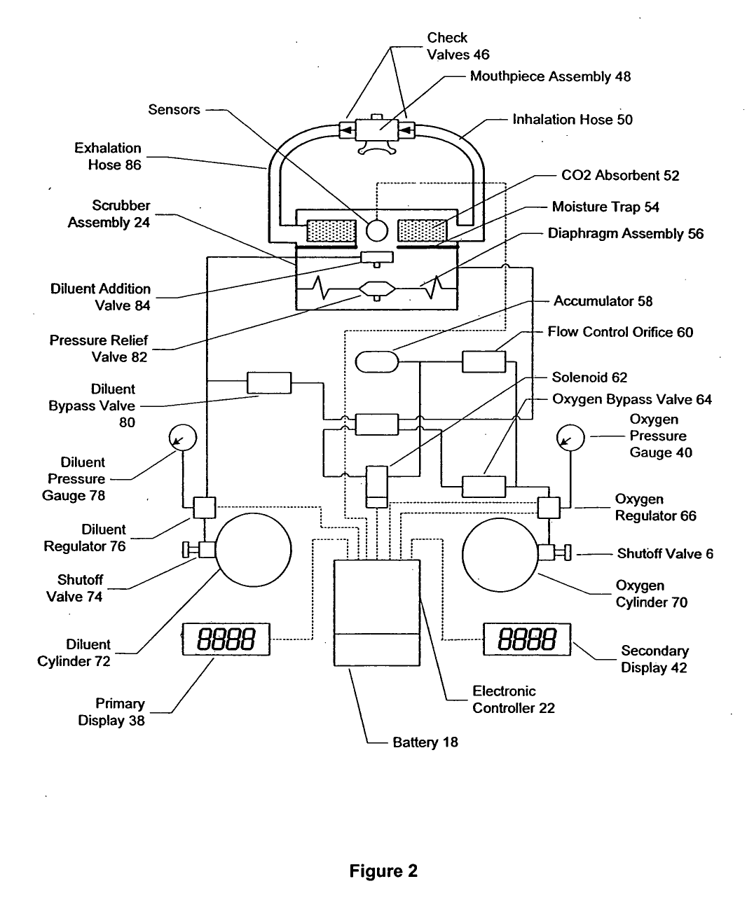

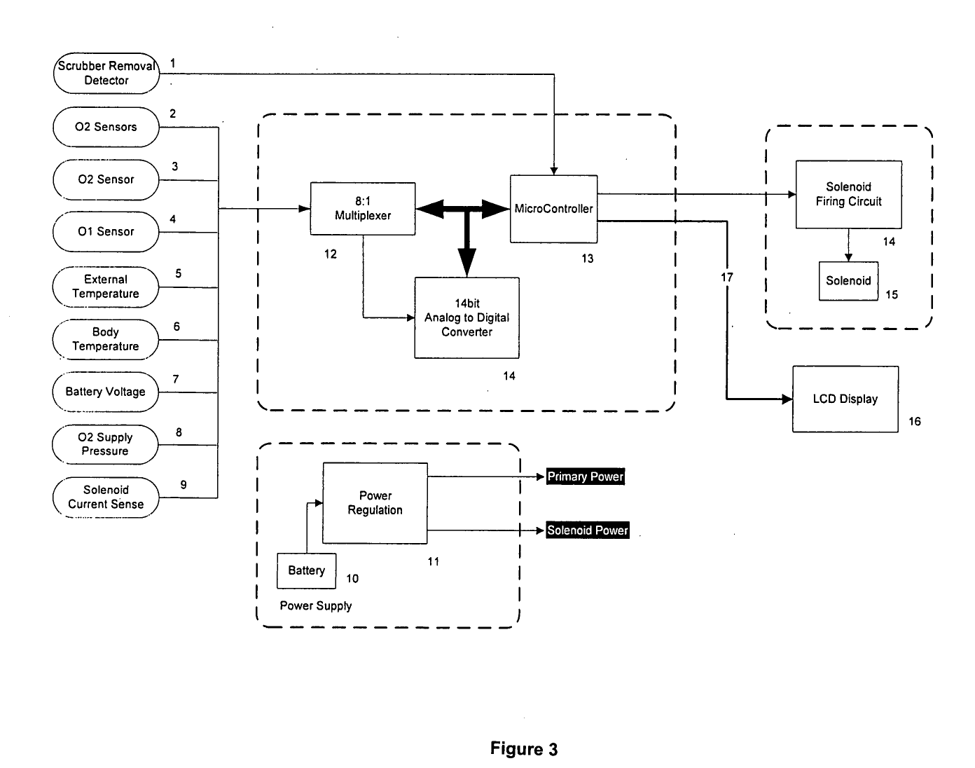

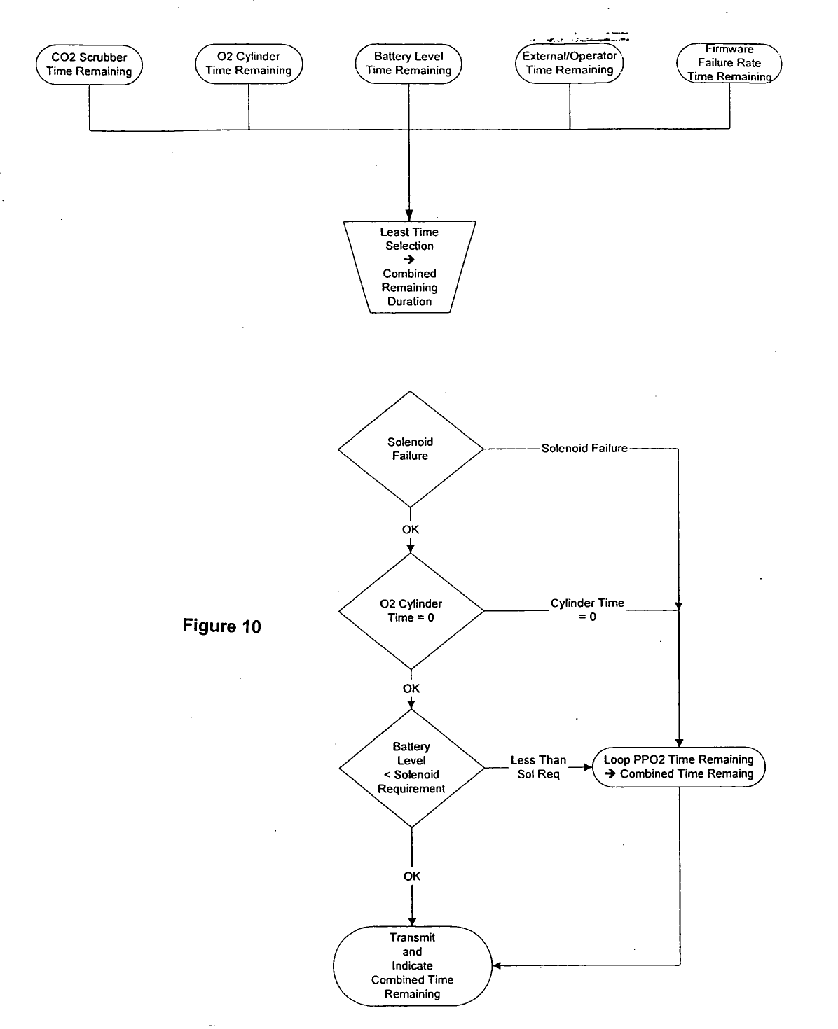

The preferred embodiment will be demonstrated as a monitor/control system such as would be found in a fully closed mixed gas rebreather system as shown in FIGS. 1 and 2. The electronic control system (FIG. 3) in this example resides in the primary electronics pod (FIG. 1-28). Many other configurations are possible and practical both for the ability to demonstrate the objects of the invention as well as for showing functionality in other types of breathing systems.

The example system combines a number of direct and indirect parameters available for detection and measurement by the microprocessor based electronics, determines a time-based factor for these parameters and then combines them into a single time remaining factor. These parameters consist of the Partial Pressure of Oxygen (PPO2) within the breathing loop (FIG. 3-2, 3, 4), the control system battery level (FIG. 3-7), the external temperature (FIG. 3-5), the operator body temperature (FIG. 3-6), the timed scrubber usage, the Firmware Failure rate, and the O2 cylinder gas pressure(FIG. 3-8). The control functions of the example electronic system relative to the said breathing system will be a solenoid (FIG. 3-15) for low pressure Oxygen addition to the breathing loop as well as a wrist mounted, cable connected LCD digital display (FIG. 1-1, FIG. 2-9, FIG. 3-16). The hardware of the control/monitor system in this preferred embodiment consists of a microprocessor such as a Motorola MC68HC908JL8CDW (FIG. 3-13) [MC68HC908JL8CDW-ND as ordered through Digikey distribution], a high resolution (34 bit) Analog-to-Digital converter such as a Maxim MAX32555ETL [as ordered direct from Maxim-IC.com] (FIG. 3-18), a Maxim 8:1 analog mux such as a MAX4783EUE [as ordered direct from Maxim-IC.com] (FIG. 3-12), a standard 3.3 volt regulator such as is manufactured by Toko TK73733SCL [TK73733SCL-ND as ordered through Digikey Distribution] (FIG. 3-11), and such industry standard capacitors, resistors, and connectors as are required. The overall circuit is powered by a standard 9 v battery (FIG. 3-10).

For the purposes of this preferred embodiment description, we will be describing a circuit with the ability to sense the partial pressure of Oxygen within a breathing loop using standard Oxygen partial pressure sensors such as Teledyne R22Ds (FIG. 3-2, 3, 4) [available from Oxycheq.com]. The controller also has the ability to control low pressure (standard SCUBA interstage pressures of between 165 psi and 95 psi) Oxygen with a solenoid such as the Wattmiser model by SnapTite [2W12w-1NB-V0A4 distributed by FasanAll] (FIG. 3-15). Additional sensor inputs are provided by a ground connected current sense resistor (FIG. 3-9) in series with the solenoid current path, a industry standard high pressure sensor in connection with the high pressure Oxygen (FIG. 3-8) and Diluent cylinders, and a temperature sensors such as a STMicroelectronics LM335 (FIG. 3-5, 6).

The circuit components are connected together in such a way (using industry standard printed circuit board techniques such as created with ORCAD Capture/Layout and ordered through PCBPRO.com) such that the 8 channel analog multiplexer (FIG. 3-12) is connected to a resistive divider which provides a battery level measurement (FIG. 3-7), the outputs of the solenoid current sense resistor (FIG. 3-9), the 3 Oxygen partial pressure sensors (FIG. 3-2, 3, 4), the temperature sensor (FIG. 3-6, 7), and the high pressure sensor output (FIG. 3-8). The output of the multiplexer (FIG. 3-12) is in turn connected to the ADC (Analog to Digital Converter) (FIG. 3-18). The digital controls of both the analog multiplexer (FIG. 3-12) and the ADC (FIG. 3-18) are connected to the microprocessor as is the circuit (solid state relay such as IR PVN012) (FIG. 3-14) which fires the gas addition solenoid.

In further addition, the circuit also provides a digital connection (FIG. 3-17) through a cable to a wrist mounted LCD display (FIG. 1-1, FIG. 1-9, FIG. 3-16) capable of displaying numeric time information.

In normal operation as pertains to the claimed and exampled art, the various sensor informations are regularly sampled and assessed and converted to a direct or indirect time factor as follows:

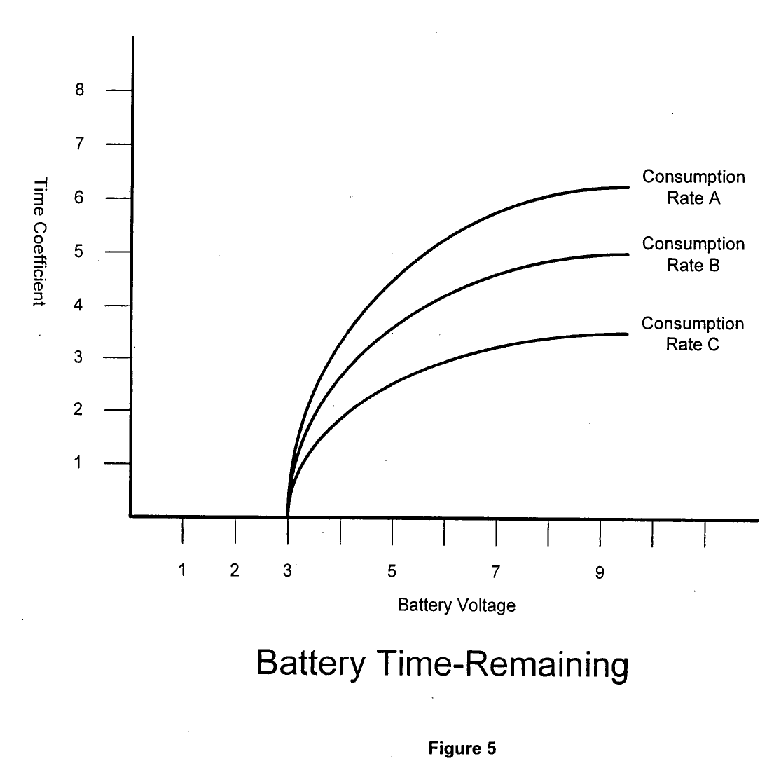

Battery Level (FIG. 5): Battery level is measured as a voltage and compared to a standardized table of 9 v alkaline battery capacity remaining relative to voltage. This capacity is then compared to capacity per time requirements for the overall system and to the current rate of use of variable capacity requirements such as the solenoid firing rate. These inputs are then calculated to yield a prediction of a time remaining relative to the battery functioning.

Power Supply Level: The power supply is also monitored independently from the battery and separately assessed as to the ability to operate the solenoid and reliably operate the microelectronics. Time factors are determined for both depending on operating threshold levels as well as anticipated rate of use should the power supply output fall to less than nominal.

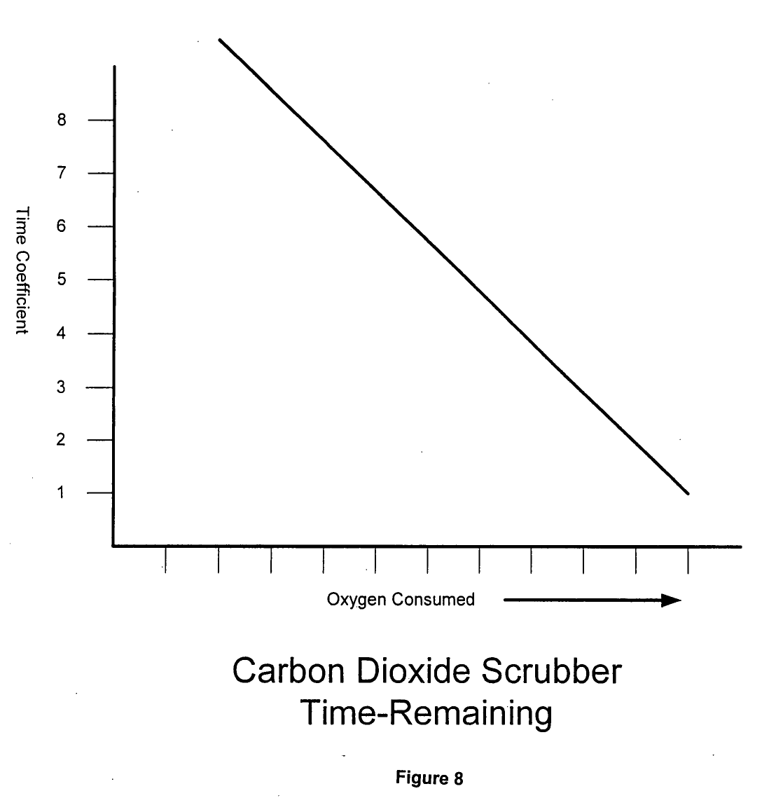

CO2 Scrubber Capacity (FIG. 8): Scrubber capacity is calculated in this demonstration of embodiment as a timed capacity relative to amount of use starting from an action of sensed scrubber replacement and increased according to the tracked amount of Oxygen used by the system according to solenoid firings and/or Oxygen Cylinder pressure usage. The net result is a time factor that tracks the amount of CO2 scrubber capacity remaining.

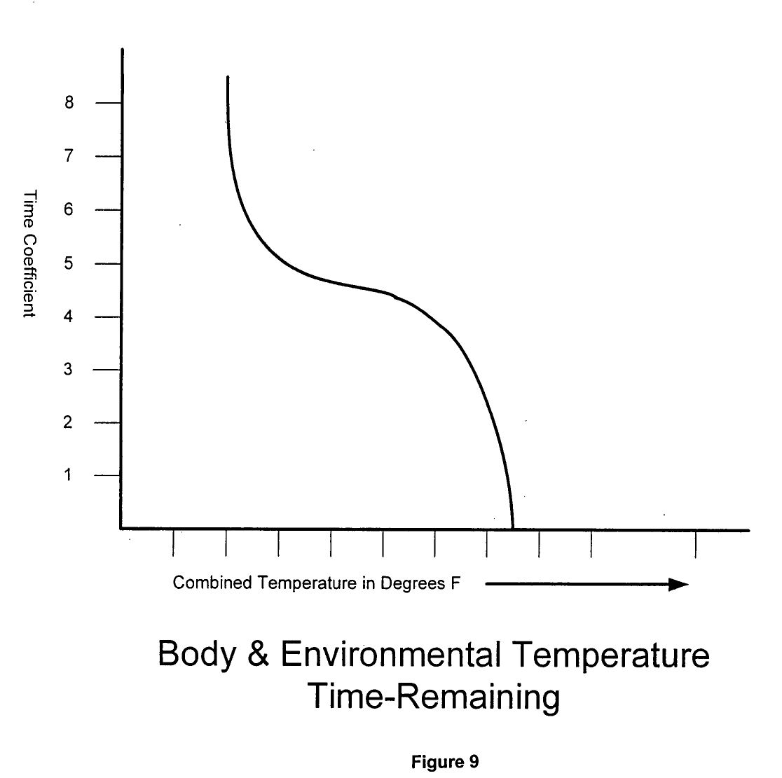

External/Operator Temperature (FIG. 9): The external and operator temperatures are measured on a regular basis and a time remaining factor relative to specified operator endurance and safety levels is calculated from comparisons to relevant pre-determined tables according to the standards requested or required pertaining to the specific industry and realm of operation.

Solenoid Operation: The Solenoid operation is sensed by means of a current sensing resistor. In the simplest embodiment, this is a go/no-go detection that determines if the solenoid has been electrically enabled as intended or not when fired. When proper functioning is detected, there is no effect on existing time factors. When improper or no functioning is detected, it is assumed that there is no Oxygen being added to the loop and the dominate time remaining factor now becomes the O2 breathing loop partial pressure factor.

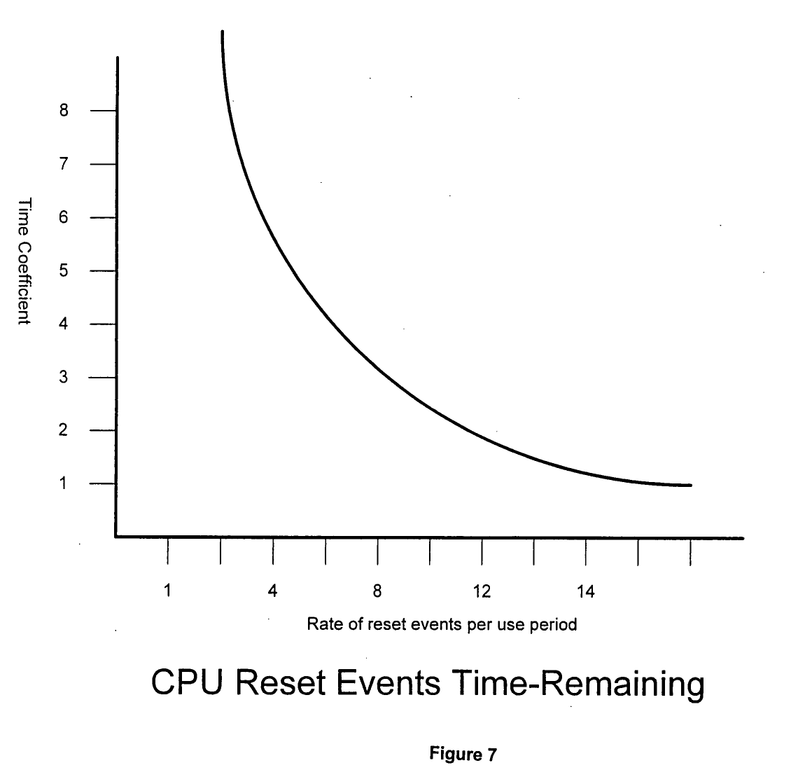

Firmware Failure Rate (FIG. 7): The firmware and microprocessor hardware design is such that unexpected deviations from normal firmware or microprocessor behavior have the expected result of forcing a reset trap within the microprocessor system. Regardless of the reasons for this type of failure, these failures are then trapped and recorded in non-volatile RAM and the program and processor are reset and restarted. If these errors occur beyond an acceptable rate threshold, a time remaining factor is created that is combined in consideration with the other time remaining factors. Typically, this is a dominate factor depending on the rate of failures. At the maximum acceptable rate of reset failures, the system defaults to the time remaining as determined by the loop PPO2 in the anticipation of no additional Oxygen being added in for that state of failure.

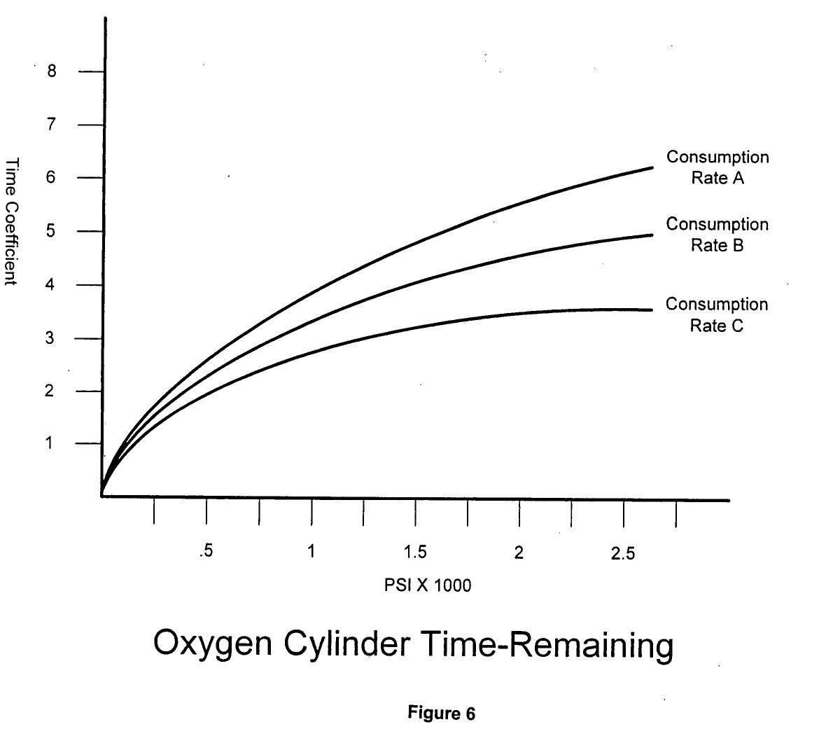

The firmware tracking of such errors depends on both hardware and firmware error traps. Hardware reset is triggered by a failure of the firmware code to execute a hardware counter reset within a pre-set time. Firmware reset is caused by hardcoded trap-jumps placed at the physical beginning of each code section. This structure prevents a run-away code execution outside of normal programmed paths. The trap-jumps execute a Flash-Memory stored record of the failure. In addition, time of operation is also regularly recorded. Each reset causes an evaluation of the reset, an increment of tracking counters, and a record made of run-time to date. These are then evaluated according to both absolute counts, absolute counts per run time, and rate of occurrence during the previous 5 and 10 minute and total operational run time. As these counts and rates increase, available time is accordingly decreased thus providing a conversion from a typically non-time based parameter into an effect on operational time. These considerations and conversions are only applicable to recoverable errors. Non-recoverable errors (complete microprocessor failure) drive the display electronics to execute an automatic warning of No Time Remaining. O2 Cylinder Pressure (FIG. 6): The O2 Cylinder Pressure time factor is calculated on the basis of current pressure as it relates to known cylinder capacity, rate of O2 addition from the known solenoid firing, and rate of actual decrease of cylinder capacity as tracked and measured by O2 cylinder pressure measurements. These factors are combined in standard methodologies to produce an estimated time remaining factor.

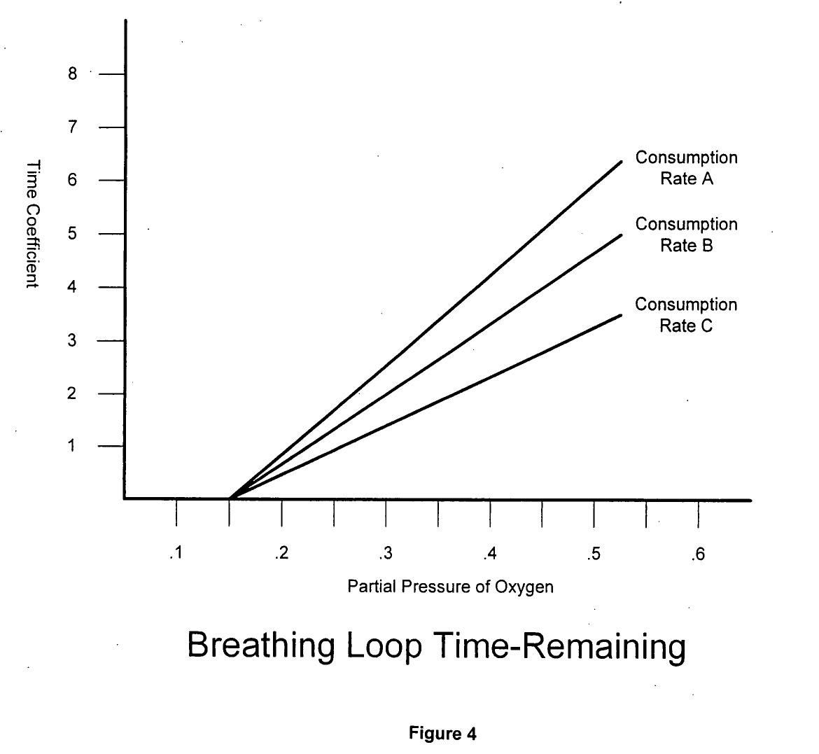

O2 Breathing Loop Partial Pressure (FIG. 4): The time remaining factor derived from this parameter is only valid when it is assumed that there is no additional Oxygen being added to the loop. In that case, the know volume of the loop is used to calculate the amount of time remaining based on both the measured historical rate of Oxygen usage as well as the measured actual rate of decline of PPO2 within the breathing loop. The time is estimated from current Oxygen levels to a predetermined level that is deemed to give a minimum amount of time to allow the user to remain conscious while changing to a backup or other means of life support. For the purposes of this embodiment, that lower level will be considered to be 0.18 Partial Atmospheres of Oxygen.

The Partial Pressure Oxygen sensors (3A, 3B, 3C) are unique among the checked parameters in that the total valid measurement is a combination of these three sensors rather than the usual determination by one sensor for other parameters or other embodiments. Each set of individual PPO2 measurements obtained and averaged. At that point, the three measurements are assessed in comparison to each other and an overall average is determined according to that comparison which determines a conservative, medium, and liberal combined results. The most conservative result is used for alarm purposes if the embodiment is so enabled. The medium is used to drive the solenoid and the liberal is used for display purposes. In normal operation, these values are the same. As one or more sensors deviate by increasing amounts from each other or the measurement channel or sensor fails, the results of these parameters vary according to purpose. The medium is designed to allow increasing failure of the Oxygen sensors to drive the solenoid according to the greatest likelihood of error in the direction of increasing the Oxygen level over the desired level. The display is always driven in the direction of any error resulting in a report of lower Oxygen levels than actual. In this way, the user is informed in a conservative manner should the sensors be functioning at a less than optimum performance while the solenoid errs on the side of too much oxygen.

In total, these above time remaining factors are then analyzed and independent factors are compared to select the least time remaining as the dominant factor (FIG. 8). In addition, indirect factors such as failure rates or solenoid functioning are examined which may direct that such additional factors as may then be appropriate be considered for the determination of the overall time remaining factor.

In this example, this factor is then computed as appropriate for display on a wrist mounted LCD device (3P) which displays to the user a single combined time remaining or combined-duration indication which allows the user to function with a much lower level of self-assessment and calculation than would otherwise be necessary for the operation of said breathing system. The LCD driver electronics incorporates an automatic reset circuit which drives a No-Time-Remaining (bailout) indication should the microprocessor fail to update the display on a regular basis.

An advantage of this embodiment is the single display is now capable of displaying multiple relevant parameters in the single display. Since this display is now independent of the specific basis, the operator's workload is decreased by not having to either translate multiple parameters into time and does not have to personally process and consider the effects of multiple parameters when the net effect of the meaning of these parameters is ultimately the same factor dealing with the remaining amount of time the breathing system is capable of functioning sufficiently enough to sustain life.

An extension of this example is a further simplification to a single LED indicator such that it could be used in either a wrist or face mounted display. A firmware algorithm is used to translate the time remaining into several states that are indicated by distinctly different display patterns. For the purposes of this example, the usable time on the breathing system is divided up into 4 categories. The categories can be expressed as Normal (from 100% capacity to 20 minutes remaining-indicated with a slow blink), Short Time (20 minutes to 5 minutes-indicated with a double blink), Very Short Time (5 minutes to 0 minutes-indicated with a triple blink), and No Time Remaining (indicated with a very fast blink).

A further extension of this example is the use of a multiple LED bar graph such as found in a MK15 type primary display which displays a number of LEDs (6 in total) in proportion to the amount of time remaining with the first LEDs green in color, followed by yellow, followed by red with the remaining red LED enabled to flash very quickly as a further indication of a warning indication of the last time state and the last two LEDs flashing in a slower state as an indication of the next longer time remaining state, followed by the next longer time state being indicated by the remaining three LEDs flashing slowly to indicate that state. All remaining states indicated by all appropriate LEDs on solid and the time state indicated solely by the number of LEDs in the ON state.

A further extension of this example is the use of wireless transmission technology to transmit the time remaining indication to the user display and/or a more remote monitoring station.

The firmware for accomplishing the above tasks is written in assembly language and downloaded using standard industry programming devices specific for the processor of choice. The firmware is structured in a number of extensible code spaces divided between interrupt driven timed structures and loop driven structures. The time driven structures provide timed standard code spaces with the time intervals occurring at 200 us, 10 ms, 50 ms, 100 ms, 1 sec, 10 sec, 1 minute, and 1 hour. The loop driven structures are divided between a Primary Loop and 2 Round-Robin Loop spaces. All code spaces in the Primary Loop space are executed through the entire loop space as frequently as possible but without regard to exact time. One of the Round-Robbin code spaces is executed once per pass of the Primary Loop code space and so are used for less time critical applications. The overall code structure is divided between 3 levels of functions dealing with Core, Standardized Support, and Application Specific code functions—all code in those spaces executing in one of the above mentioned timed or loop driven code spaces. Each of the measurement functions is carried out on a timed and table driven process which accumulates one set of measurements every 50 ms. As each measurement is selected, the multiplexer is set to pass that measurement parameter through to the ADC (Analog to Digital Converter), the ADC is then instructed to make the measurement which is then stored in RAM in the microprocessor. This is a Round-Robin process initiated by a timer in the 50 ms code space. Execution Flags are set as each measurement is taken to cause an additional Round-Robin process to execute which averages the value of each measurement and determines if the measurement is valid in terms of ADC functionality.

Secondary backup systems may also be considered as an extension of this art for example in a system which may have an additional monitor circuit or other form of microprocessor redundancy.

We claim:

1. An electronic system for closed or semi-closed rebreathing devices comprising a microcontroller, inputs and sensors, display output device and power supply, wherein the microcontroller calculates the remaining usable system time based on at least two sensed inputs within the breathing system with a determined rate of use time factor for each input.

2. An electronic system for land based SCBA (Self Contained Breathing Apparatus) devices comprising a microcontroller, inputs and sensors, display output device and power supply, wherein the microcontroller calculates the remaining usable system time based on at least two sensed inputs within the breathing system with a determined rate of use time factor for each input.

3. An electronic system for water based SCUBA (Self Contained Underwater Breathing Apparatus) devices comprising a microcontroller, inputs and sensors, display output device and power supply, wherein the microcontroller calculates the remaining usable system time based on at least two sensed inputs within the breathing system with a determined rate of use time factor for each input.

4. The system of claims 1, 2, and 3 wherein a single common system of minimum indication that uses the relevant parameters specific to different types of breathing systems is such that an operator may switch between different types of systems with the use of similar, simple information systems dealing only with time remaining on the system.

5. The system of claims 1, 2, and 3 wherein the microcontroller calculates the remaining usable system time additionally on at least one sensed environmental factor.

6. The system of claims 1, 2, and 3 wherein the microcontroller calculates the remaining usable system time additionally on at least one sensed biometric factor.

7. The system of claim 1 wherein a sensed input within the breathing system includes remaining carbon dioxide scrubber absorption capacity.

8. The system of claim 1 wherein a sensed input within the breathing system includes the measurement of Oxygen partial pressure within the breathing loop of the rebreathing system.

9. The system of claim 1 wherein a sensed input within the breathing system includes the sensed mechanical functionality of the solenoid.

10. The system of claim 1 wherein a sensed input within the breathing system includes sensed CO2 levels.

11. The system of claims 1, 2, and 3 wherein a sensed input within the breathing system includes battery level.

12. The system of claims 1, 2, and 3 wherein a sensed input within the breathing system includes the high pressure gas supply of the breathing.

13. The system of claims 1, 2, and 3 wherein a sensed input within the breathing system includes detected electronic measurement hardware functionality.

14. The system of claims 1, 2, and 3 wherein a sensed input within the breathing system includes detected electronic firmware functionality.

15. The system of 5 wherein a sensed environmental factor includes external temperature.

16. The system of 5 wherein a sensed environmental factor includes external location.

17. The system of 5 wherein a sensed environmental factor includes external pressure.

18. The system of 6 wherein a sensed biometric factor includes body temperature.

19. The system of 6 wherein a sensed biometric factor includes breathing rate.

20. The system of 6 wherein a sensed biometric factor includes heart rate.

21. The system of 6 wherein a sensed biometric factor includes blood pressure.

22. The system of 6 wherein a sensed biometric factor includes stress level.

23. An electronic monitoring system for a breathing system which has:
A power supply to power a microprocessor circuit from a battery device;
A electronic circuit to measure Oxygen Levels in the breathing gas;
A electronic circuit to measure Battery Levels in the power supply;
A electronic circuit to measure high pressure gas levels;
A electronic circuit to measure CO2 scrubber capacity;
A electronic circuit to sense electronic hardware failures;
A electronic circuit to sense mechanical breathing system conditions;
A electronic circuit to measure operator body temperature;
A electronic circuit to measure operator heart-rate;
A electronic circuit to measure external temperature;
A electronic circuit to measure external pressure;
A microprocessor to connect and control the measurement and sense capabilities;
and provide a operable basis for firmware functionality.

24. A microcode module to process measurements and sensed conditions into numeric values representing those measurements.

25. A microcode module to detect and sense firmware and microcontroller failures and rates of those failures.

26. A microcode module to convert all sensed and measured conditions into time based factors based on the affect of those factors on the remaining ability of the breathing system to provide adequate life support.

27. A microcode module to combine all time factors into a single overall time based factor which is used as a measure of remaining usable time on the breathing system.
